(19) United States
(12) Patent Application Publication
(10) Pub. No.: US 2017/0001200 A1
(43) Pub. Date: Jan. 5, 2017

(54) CANNABIS PLANT FILTER AND APPARATUS

(71) Applicant: Mitchell Leffel, Fortuna, CA (US)

(72) Inventor: Mitchell Leffel, Fortuna, CA (US)

(21) Appl. No.: 15/091,400

(22) Filed: Apr. 5, 2016

Related U.S. Application Data
(60) Provisional application No. 62/144,841, filed on Apr. 8, 2015.

(51) Int. Cl. B02C 23/14; B02C 23/10; B07B 1/28; A61K 36/185

(52) U.S. Cl. CPC B02C 23/14; A61K 36/185; B02C 23/10; B07B 1/28; A61K 2236/15; B07B 2201/04

(57) ABSTRACT

An assembly for separating cannabis trichomes by a vibrating platform and at least one filter.

This application claims benefit of priority to U.S. Provisional Patent Application No. 62/144,841, entitled “Cannabis Plant Filter and Apparatus,” filed Apr. 8, 2015; the aforementioned application being incorporated by reference in its entirety for all purposes.

### TECHNICAL FIELD

The disclosed embodiments generally relate to a device for mechanically harvesting cannabis plants.

### BACKGROUND

Cannabis plants can be harvested and processed to obtain numerous kinds of chemical compounds used in medical products. For example, the compound tetrahydrocannabinol (THC) can be extracted from cannabis plants. In particular, THC can be obtained by refining micron-sized outgrowths on the cannabis plant called trichomes. Trichomes may vary in size (e.g., between 60 to 200 microns).

There exist meshes and screens having openings which are approximately trichome-sized.

There further exist machines to harvest cannabis plants by mechanically cutting the plant apart.

### BRIEF DESCRIPTION OF THE DRAWINGS

FIG. 1 illustrates a trichome filter device, according to an embodiment.

FIG. 2 illustrates a cannabis harvester including a trichome filter device, according to an embodiment.

FIG. 3 illustrates an apparatus for harvesting cannabis plants, according to an embodiment.

FIG. 4a-4c illustrate examples of a drum assembly for use with the apparatus of FIG. 3, according to an embodiment.

FIG. 5 illustrates a removable lid for a cutting assembly, according to an embodiment.

FIG. 6 illustrates a housing for a filter device according to an embodiment.

### DETAILED DESCRIPTION

Embodiments described herein include an assembly for separating trichomes, comprising a vibrating platform to receive trichomes, and at least one filter to separate the trichomes.

In an embodiment, a solventless filtration device or system includes a series of filter layers. A trichome will pass through layers having openings which are larger or equal-sized to the trichome, and will be stopped by smaller-sized openings. The layers are positioned so the size of the openings gradually decreases per layer. Trichomes are introduced to the device at the first layer (having the largest openings) and pass through the remaining layers to the last layer (having the smallest openings). Each layer filters out trichomes which are larger than the size of its openings, but retains trichomes which are smaller or equal-sized with the opening size of the prior layer.

In examples, the filtration device includes a mechanism to move trichomes through the layers. In an example, a vibration mechanism shakes the filtration device to sift clumps of trichomes through the openings.

In an aspect, the filtration device includes a removable drawer. Filters are stacked in the drawer to capture trichomes. Filters nearer the bottom of the drawer capture finer trichomes, and filters nearer the top of the drawer capture larger trichomes.

Among other benefits, the filtration device divides trichomes by size without use of a chemical solvent. Advantages include a simplification of the number and kinds of components in the device; no exposure of trichomes or a user of the device to chemicals; and simpler incorporation of the device into assemblies for harvesting cannabis plants.

In an example, a trichome filtration device is positioned beneath a cannabis plant harvester. The harvester separates the cannabis plant into parts for processing. Trichomes detach from the plant and are recovered by the filtration device. The filtration device separates the trichomes by size.

In an embodiment, the filter device is incorporated into a mobile apparatus for removing cannabinoid-rich parts of a cannabis plant. The apparatus includes a removable cutting assembly within the apparatus which operates to mechanically remove parts from the cannabis plant. The apparatus includes a removable cover (e.g., lid) to access the cutting assembly and/or filter device.

Trichome Filter

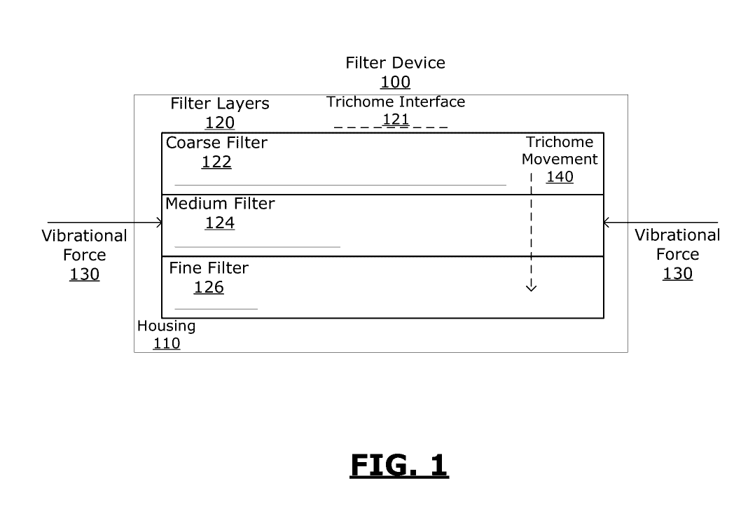

FIG. 1 illustrates a trichome filter device, according to an embodiment. As trichomes pass through a series of filter layers, they are collected and sorted by their size. The filter apparatus is vibrated to facilitate movement of the trichomes through the filters.

In the illustration of FIG. 1, filter device 100 includes a housing 110 surrounding filter layers 120. While three filters (coarse filter 122, medium filter 124 and fine filter 126) are shown in the illustration of FIG. 1, the number of layers is illustrative and embodiments provide for filter layers 120 to include any number of layers.

Suitable filter designs for filter layers 120 include, without restriction, screens, porous mats, and meshes, or other surfaces to selectively retain trichomes. Trichomes will be collected based on the filter's openings' size. For example, coarser filters (e.g., filters having relatively large openings) will allow a wider range of trichome sizes to pass through than finer filters (e.g., filters having relatively small openings). Coarse filters will also tend to remove non-trichome impurities; for example, coarse filters may be used to exclude flowers or leaves from later filtration.

In the example of FIG. 1, each filter layer has openings having substantially similar size. Filter layers 120 comprise a coarse filter 122 having relatively large openings; medium filter 124 has medium-sized openings; and fine filter 126 has relatively small openings.

The openings of filter layers 120 approximate the sizes of trichomes. In an example, the coarse filter includes openings having a diameter of approximately 200 microns, and allows trichomes to pass through while filtering non-trichome impurities; the medium filter includes openings having a diameter of approximately 120 microns, to retain large trichomes; and the fine filter includes openings having a diameter of approximately 60 microns.

When trichomes enter filter device 100 for filtration (e.g., via trichome interface 121), they pass through the filters in the direction of trichome movement 140. In the illustration of FIG. 1, the filters are stacked vertically and the trichomes move downwards via gravity. The remaining trichomes and/or microscopic impurities which pass through fine filter 126 are then stored within housing 110 (not shown).

Embodiments recognize that the regular flow of trichomes through filter layers 120 affects the speed of filtration, and further recognize that the openings in the filter layers 120 may be blocked by large trichome and non-trichome particles that will not pass through. Furthermore embodiments recognize that the usage of chemical solvents (e.g., butane, carbon dioxide) to transport trichomes through the filter layers 120 include disadvantages such as increases in cost, reduced mobility, and potential damage to the trichomes.

In the illustration of FIG. 1, vibrational forces 130 cause the filter layers 120 to move. Examples of movements caused by vibrational forces 130 include shaking, oscillation, and churning. Among other advantages, this facilitates movement of trichomes through openings, clears blocked openings, breaks clumps (e.g., trichomes stuck to the filter layer) to break away.

In an implementation, filter device 100 is provided as part of an apparatus for harvesting cannabis plants. The apparatus's movement during operation produces vibrational force 130 on filter layers 120.

Housing 110 may be designed to have a form factor for use with other devices. For example, housing 110 may be designed to compactly fit within a cannabis plant separation device. In an example, as shown in FIG. 6, housing 110 is designed as a drawer.

In an example such as illustrated by FIG. 1, housing 110 may be configured so that each filter layer may be individually removed from the housing 110. For example, an individual filter can be repaired, maintained, cleaned and/or have its trichomes removed, without removal of the other filters from the housing.

In an implementation, the top filter (e.g., coarse filter 122 as illustrated in FIG. 1) comprises a mesh mat, and the trichome interface 121 is an area of the mat on which trichomes are placed. In another implementation, the filter device 100 is provided underneath a cannabis plant harvesting apparatus, and the trichome interface 121 connects the harvesting apparatus with the filter device. As the harvesting apparatus breaks apart the cannabis plant, trichomes disassociate from the plant and enter the filter apparatus.

In implementations, trichome interface 121 receives both trichomes and trichome-rich cannabis plant parts (e.g., cannabis leaves). The coarse filter retains these trichome-rich parts. As the filter device 100 shakes or moves from vibrational force 130, trichomes may further detach from the trichome-rich parts and move through the filter layers 120.

Cannabis Separation Device

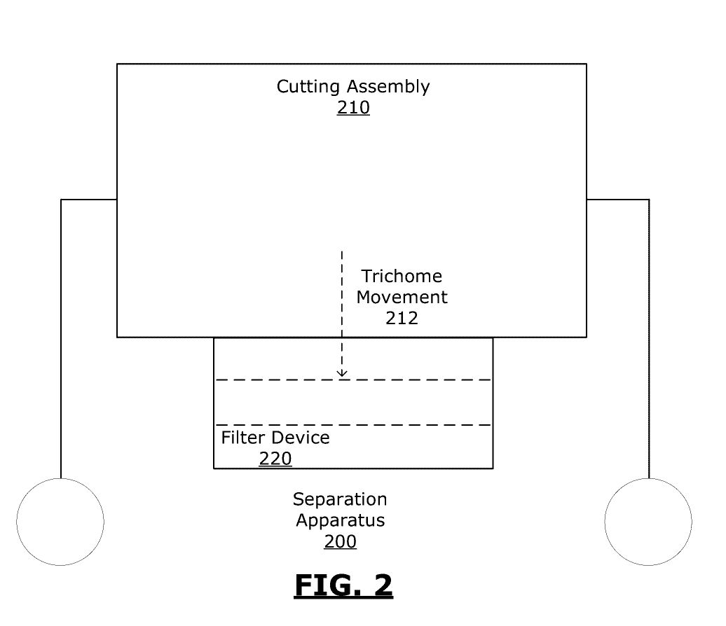

FIG. 2 illustrates a cannabis harvester including a trichome filter device, according to an embodiment. A harvester as illustrated in FIG. 2 may be implemented using structures and features as illustrated in the example of FIG. 1. Reference is made below to the components illustrated by FIG. 1 in describing FIG. 2.

Separation apparatus 200 cuts cannabis plants into different plant parts within cutting assembly 210. Trichomes from the cannabis plants are filtered and stored in filter device 220. Harvesting machine 200 is constructed from material (e.g., medical grade stainless steel or aluminum) for storing the cannabis plant parts. In an example, filter device 220 corresponds to trichome filter device 100 of FIG. 1. In an implementation, separation apparatus 200 is a harvesting machine 200, and cutting assembly 210 is a rotating drum.

Drum 210 includes structures (e.g., cutting implements) to grind, cut or separate the cannabis plant in substantially intact portions. The intact portions of the cannabis plant can include stalks, leaves, and flowers. One action which can result from the drum 210 includes separation of flowers or leaves from stalks. Thus, an output of the drum 210 includes separated components of the cannabis plant, cut portions of the cannabis plant (e.g., stalk, flower, leave), and/or cannabis debris (collective “cut cannabis matter”).

According to one embodiment, the drum 210 feeds or moves the cut cannabis matter to the filter device 220. As described with other examples, the cut cannabis matter can include trichomes, and certain portions of the cut cannabis matter can be trichome-rich. The cut cannabis matter is directed actively (e.g., by mechanical force) or passively (by gravity) into filter device 220 for further processing and manipulation. To direct cut cannabis matter, the drum 210 can include channels, chutes, openings, or transport devices (e.g., robotic arm) to collect cut cannabis matter at the filter device 220. Alternatively, the cut cannabis matter can be collected at an underside platform of the drum 210 separately from the filter device 220, and then moved to the filter device 220. Furthermore, harvesting machine 200 may include mechanisms which operate in a manner (e.g., a rotating drum) which shift the contents of the drum 210 towards the filter device 220.

According to an aspect, drum 210 and/or filter device 220 are positioned to facilitate the trichome movement 212 through the harvesting machine 200. In the illustration of FIG. 2, drum 210 is placed above filter device 220. Trichomes separate from the cut cannabis matter within the drum 210 and aggregate directly onto the filter device 220 via gravity, where filtering can occur using gravity and movement.

The harvesting machine 200 may include storage areas to store parts of the cannabis plant removed by drum 210 and/or filtered by filter device 220. For example, drum 210 may include a drum to store cannabis flowers and large branches.

Harvesting machine 200 may include access features to provide a user with access to the different structures of the separation device. In an example, a removable lid enables a user to access structures within the drum 210. Furthermore, in examples the individual parts of drum 210 and/or filter device 220 are separable from each other.

In implementations, harvesting machine 200 includes portability features which facilitate movement of the device. For example, harvesting machine 200 may include a wheeled frame.

Drum 210 may be configured to physically prevent branches, flowers and other large pieces of the cannabis plant from entering the filter. For example, openings within drum 210 may be large enough to permit trichomes and trichome-rich cannabis leaves to move into filter device 220, but small enough that flowers of cannabis plants do not move into the filter device.

FIG. 3 illustrates an apparatus for harvesting cannabis plants, according to an embodiment. In particular, an apparatus as illustrated in FIG. 3 may be implemented using structures and features as illustrated in the examples of FIGS. 1 and 2. Reference is made below to the components of FIGS. 1 and 2.

Drum 310 and mount 330 form a cutting assembly (e.g., with reference to FIG. 2, cutting assembly 210). Cannabis plants are placed in drum 310. Drum 310 rotates and mechanically separates the cannabis plants. As drum 310 operates, trichomes and trichome-rich parts of the cannabis plant detach. The trichomes and trichome-rich parts move into and through filter device 320. Filter device 320 retains the trichomes and organizes them by size.

During operation of the apparatus, drum 310 trichomes may be loosened from the plant in various manners. For example trichomes may fall off the plant due to motion of the drum; due to the force of the cutting assembly mechanically separating the plant; or by vibrational forces on the drum 310 or apparatus 300. Fallen trichomes are recovered and separated by filter device 320. Drum 310 includes features (e.g., apertures to the filter device 320) which enables the movement of plant parts having trichomes to the filter device, while minimizing unwanted plant parts. The rotation of drum 310 shakes the contents of the drum to minimize blockage of any such features.

Drum 310 and/or mount 330 each include implements to separate cannabis plants. For example, drum 310 includes cutting blades in its interior which cut the plants as the drum 310 rotates.

Apparatus 300 may include a programmable feature (e.g., an electronic processor coupled to a drum control mechanism) to control operation of the drum. For example, a processor may be programmed to rotate the drum at a fixed speed (e.g., 16 revolutions per minute), or for a fixed period of time. During rotation, mount 330 stabilizes the drum.

Power source 350 provides electrical energy for the functioning and control of apparatus 300. In the embodiment of FIG. 3, power source 350 comprises an external motor. While the power source 350 may also be located internally to apparatus 300, an external power source as illustrated in FIG. 3 provides advantages, such as easier repair and maintenance; improved safety; protection of the cannabis plant within the drum from pollutants emitted by the power source; and enhanced functionality of the filter device 320.

As described regarding FIG. 1, vibrational forces assist in relieving congestion within the filter device by moving trichomes on and through the filter layers. As illustrated by FIG. 3, power source 350 may positioned to provide vibrational forces to filter device 320. Operation of power source 350 causes filter device 320 and its corresponding filter layers to vibrate during operation of apparatus 300. These vibrational forces may be enhanced by placement of the power source 350 at least proximate to filter device 320. Vibrational forces may also be provided by other parts of the apparatus 300 (e.g., drum 310) or a separate vibrational mechanism.

In an aspect, the apparatus of FIG. 3 includes a mobility feature (wheeled frame 360) to improve portability of apparatus 300. In addition to improving the mobility of apparatus 300, the frame improves the stability of apparatus 300 during operation.

Removable lid 340 provides a user with access to the drum 310 and other parts of apparatus 300, and prevents the cannabis plant and its pieces from being ejected from the apparatus during operation. Additionally the removable lid 340 acts as a safety feature for a user during device operation by preventing access to the implements for separating the cannabis plant within drum 310 and/or mount 330.

A user may remove the removable lid 340 to place cannabis plants in drum 310, and replace the lid to operate the device. The lid also is removable to remove the drum 310 from the apparatus 300.

Drum 310 may have customized implements for separating cannabis plants. For example, drums may have cutting implements that are optimized for cutting specific kinds of cannabis plant; for obtaining specifically sized parts; or for operating at particular speeds. A user of apparatus 300 who needs to replace drum 310 may remove the removable lid 340.

Drum replacement may be enhanced by modular features on the apparatus 300 or its corresponding parts. For example, drum 310 may be one of a set of drums which each have a unique modular feature that facilitates placement and removal of the drum from apparatus 300. Furthermore, mount 330 and/or removable lid 340 can include separate physical features which enable operation of apparatus 300 only when the drum has the appropriate modular feature. For example, drum 310 and other drums within a related set could include a hook to physically couple with mount 330 and/or removable lid 340. The hook prevents the drum from being coupled with other different kinds of mounts and lids.

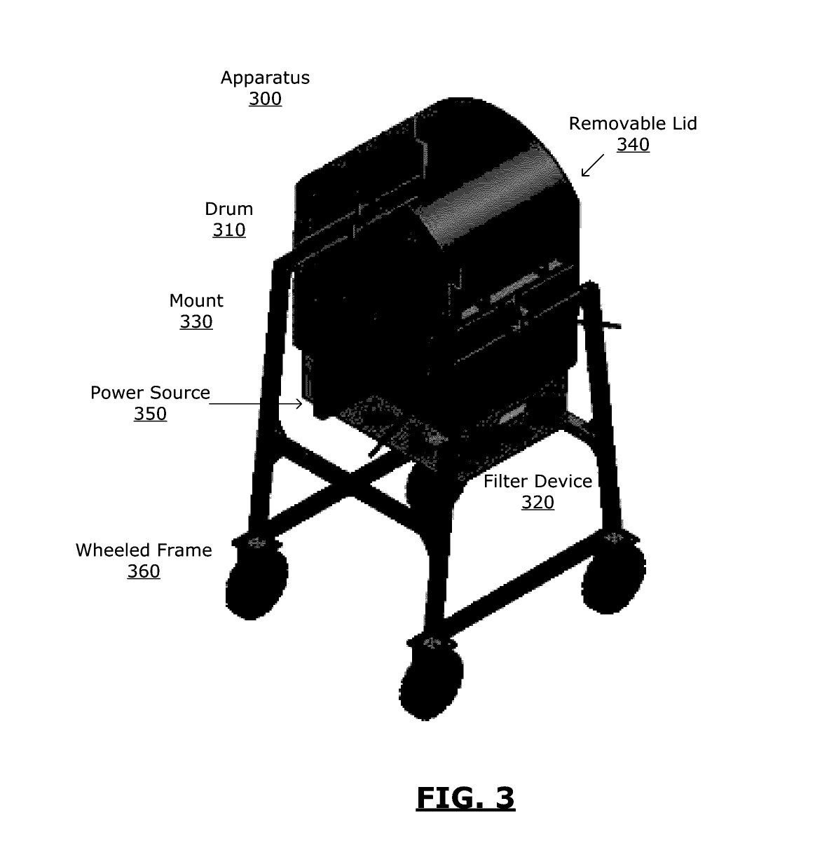

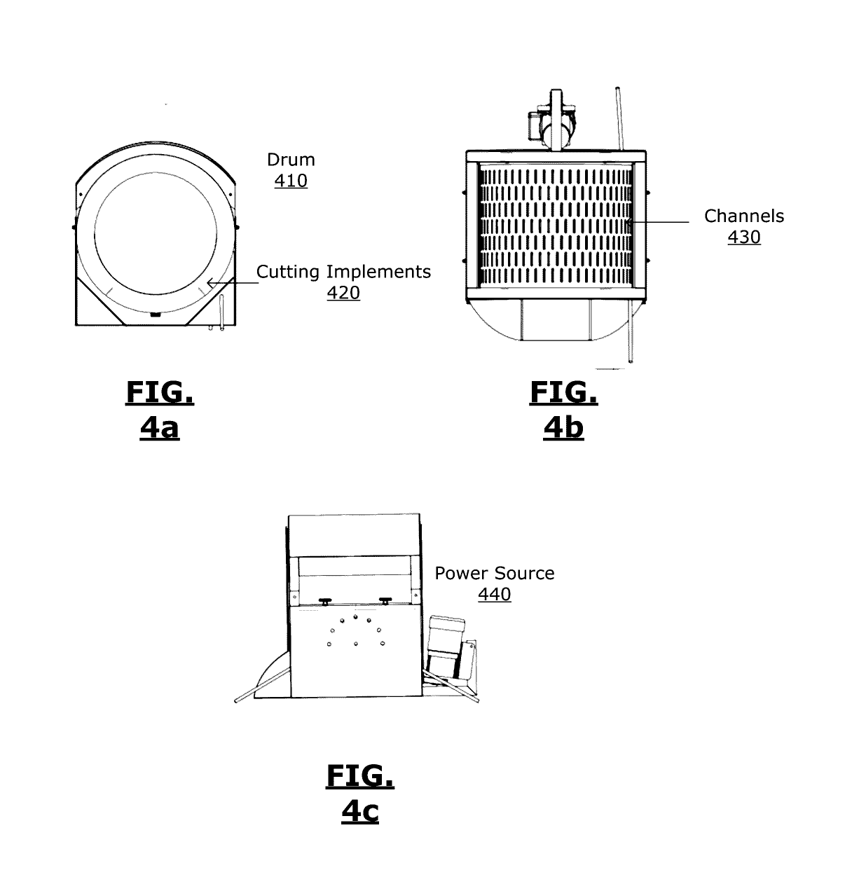

FIG. 4a-4c illustrate examples of a drum assembly for use with the apparatus of FIG. 3, according to an embodiment. In embodiments, the structures illustrated in FIG. 4a-4c correspond to structures and features illustrated in FIG. 1-3. Reference is made below to the corresponding parts of FIG. 1-3.

FIG. 4a illustrates an example where cutting implements 420 are positioned on drum 410 for removing parts of a cannabis plant. As drum 410 rotates, parts of the plant come in contact with the cutting implements 420 and are cut from the plant. Trichomes on the plant are detached during operation of the drum, and recovered by a filter device.

FIG. 4b illustrates a drum having channels 430. Channels 430 enable the passive movement of trichomes to a filter device, while impeding large pieces of cannabis plants.

FIG. 4c illustrates a drum connected to a power source 440. In the illustration of FIG. 4c the power source 440 is positioned beneath the drum and is proximate to a filter device. Vibrational forces from the drum and power source 440 cause the filter device to shake, and cause trichomes to move through and on the filter layers.

Removable Lid

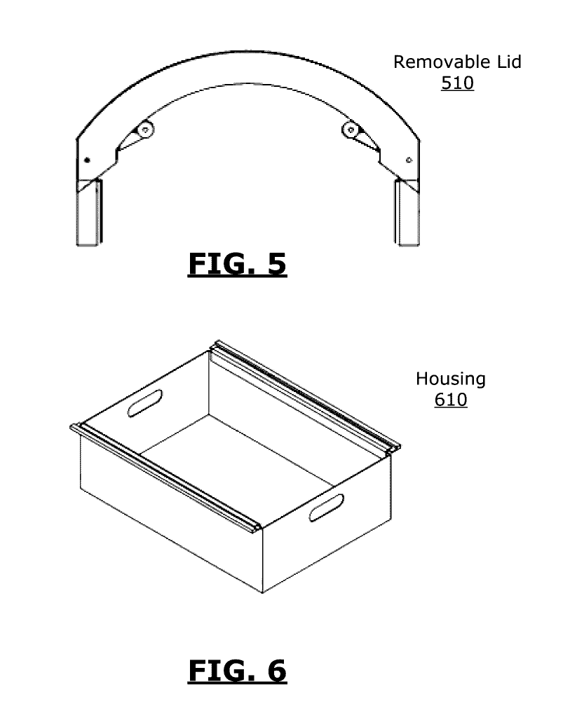

FIG. 5 illustrates a removable lid for a cutting assembly, according to an embodiment. A lid as illustrated in FIG. 5 may be placed on top of a drum and/or drum mount as described in FIG. 3. The removable lid 510 also includes safety features, such as protruding features on its rim, which securely couple with corresponding features of the drum or mount. Once coupled, the lid stabilizes the mount while in operation and prevents the contents of the drum from being ejected.

Removable 510 also includes modular features for use with drums and mounts having corresponding modular features. Modular features of the lid include features which are designed for use with a particular set of mounts or drums, so that any drum in the set may be used with the that lid.

FIG. 6 illustrates a housing for a filter device according to an embodiment. A housing as illustrated in FIG. 6 may be used with the assemblies and devices of FIG. 1-5, and reference is made to the corresponding structures of those figures in describing FIG. 6.

In the illustration of FIG. 6, housing 610 includes a drawer in which filter layers can be vertically stacked. Trichomes enter through the top layer nearest to the opening of the housing, and move through filter layers to the bottom of the housing. The drawer includes gripping features (e.g., as shown in FIG. 6, gripping slits) on its front and rear sides which a user may grip to insert and remove the filter apparatus.

Additionally the housing 610 illustrated in FIG. 6 includes securing features (rails) to secure the housing within a cannabis plant separation assembly. In the example of FIG. 6, the rails enable a user to slide the housing into and out of the cannabis plant separation assembly on the rails. The cannabis plant separation assembly includes features which grip the rails and prevent the housing 610 from being dislodged.

The rails of FIG. 6 are designed to enable the housing 610 to shift according to vibrational forces provided by operation of the device and other vibrational mechanisms, while preventing the housing from being disconnected or otherwise separated from the cannabis plant separation assembly during operation. Embodiments provide for the use of other securing features.

Although illustrative embodiments have been described in detail herein with reference to the accompanying drawings, variations to specific embodiments and details are encompassed by this disclosure. It is intended that the scope of embodiments described herein be defined by claims and their equivalents. Furthermore, it is contemplated that a particular feature described, either individually or as part of an embodiment, can be combined with other individually described features, or parts of other embodiments. Thus, absence of describing combinations should not preclude the inventor(s) from claiming rights to such combinations.

What is claimed is:

1. An assembly comprising:
a platform capable of rapid or repetitive movement, the platform including a base segment to retain cannabis leaves, the platform being operable to subject the cannabis leaves to movement and to distribute trichomes from the cannabis leaves through the base segment; and
a filter layer to receive trichomes of a variety of sizes from the base segment when the cannabis leaves are subjected to movement with the platform.

2. The assembly of claim 1, wherein the filter layer includes multiple filters, each of the multiple filters having a variety of granularity in order to separate and collect trichomes of different sizes.

3. The assembly of claim 2, wherein the filters of the filter layer include multiple filters which have different size openings from other filters of the filter layer.

4. The assembly of claim 3, wherein the multiple filters include a first filter having openings of about 200 microns, a second filter having openings of about 120 microns, and a third filter having openings of about 60 microns.

5. The assembly of claim 1, wherein each filter is individually laterally removable from the assembly.

6. The assembly of claim 1, further comprising a cutting device positioned above the platform to separate a cannabis plant.

7. The assembly of claim 6, further comprising a stainless steel housing at least partially encompassing the cutting device.

8. The assembly of claim 7, further comprising a power source positioned on an exterior of the housing.

9. The assembly of claim 7, further comprising a removable lid to access the cutting device.

10. The assembly of claim 6, wherein the platform comprises a movable drawer in which the filters are stacked.

11. A cutting assembly for separating a cannabis plant, comprising:
a rotating drum, the drum comprising a plurality of cutting implements to remove leaves from the cannabis plant; and
a filter device to receive cannabis leaves from the drum, the filter device comprising one or more filters to separate trichomes from the cannabis leaves.

12. The assembly of claim 11, wherein the filter device includes multiple filters, each of the multiple filters having a variety of granularity in order to separate and collect trichomes of different sizes.

13. The assembly of claim 12, wherein the filters of the filter device include multiple filters which have different size openings from other filters of the filter device.

14. The assembly of claim 13, wherein the multiple filters include a first filter having openings of about 200 microns, a second filter having openings of about 120 microns, and a third filter having openings of about 60 microns.

15. The assembly of claim 11, wherein each filter is individually laterally removable from the device.

16. The assembly of claim 11, further comprising a removable lid to access the rotating drum.

17. The assembly of claim 11, further comprising a power source positioned on an exterior of the housing.

18. The assembly of claim 11, further comprising a removable lid to access the drum.

19. The assembly of claim 11, wherein the filter device comprises a movable drawer in which filters are stacked.

20. A device for cutting a cannabis plant, comprising:
a cutting assembly for removing and storing parts of a cannabis plant, the cutting assembly having at least one external modular feature common to a set of cutting assemblies for cannabis plants; and
a housing configured to secure the cutting assembly by the external modular feature.
